(19) United States
(12) Patent Application Publication
(10) Pub. No.: US 2026/0364233 A1
(43) Pub. Date: Nov. 19, 2026

(54) Breast Pump

(71) Applicant: MEDELA HOLDING AG, Baar (CH)

(72) Inventors: Charles Kim, McHenry, IL (US); Kalpesh Patel, McHenry, IL (US); David Cho, Arlington Heights, IL (US); Michael Larsson, Baar (CH)

(21) Appl. No.: 19/206,244

(22) Filed: May 13, 2025

(51) Int. Cl. A61M 1/06

(52) U.S. Cl. CPC A61M 1/0693; A61M 1/064

(57) ABSTRACT

A breast pump including a breast shield adapted to at least in part receive a breast of a lactating mother or user, an aggregate for generating a cyclic suction profile for expressing milk from the breast, a milk container providing a reservoir for the expressed milk, and a controller for controlling the aggregate. The aggregate may be formed by a motor and a pump transferring the rotational movement of the motor into at least one cyclic suction profile, which suction profile includes a vacuum start phase, a vacuum application phase, a vacuum maintenance phase and a vacuum release phase, which phases are consecutive to each other and specifically designed with respect to relative duration within the suction cycle and pressure effective in respective phase to optimize the vacuum application to the breast of the lactating user with the scope to obtain an increase milk extraction.

### FIELD OF THE INVENTION

The present invention relates generally to a breast pump. Specifically, the present invention relates to a breast pump including a breast shield adapted to at least in part receive a breast of a lactating mother or user. The breast pump furthermore has an aggregate for generating a cyclic suction profile for expressing milk from the breast. The breast pump has a milk container providing a reservoir for the expressed milk and a controller for controlling the aggregate. The breast shield usually has or is in communication with a nipple tunnel providing a fluid passage for the expressed milk from the breast towards the reservoir. The aggregate may be formed by a motor and a pump transferring the movement of the motor into at least one cyclic suction profile or vacuum curve.

### BACKGROUND

Breast pumps for use by nursing mothers are well known. They allow the nursing users to express the breastmilk as necessary or convenient, and further provide collection of the breastmilk for later use. For some users, breast pumps may be a necessity, such as when the child has suckling problems, or if the user has problems with excessive or deficient milk production, or soreness, deformation, or injury of the mammilla. Of particular instance herein is the situation confronting a user where the infant is premature, and therefore is most always separated from the mother.

Electrically-driven breast pumps are commonplace, typically including a vacuum pump which has an electric motor that plugs into standard house current, and/or operates with battery power. Advantages of this type of pump are convenience, ready controllability and regulation of the vacuum, and in many instances the ability to pump both breasts at once.

Electrically-driven, motorized breast pumps generally have a driving mechanism for generating the vacuum (negative pressure) to be applied at the breast geared to a particular sequence, or curve, of vacuum or negative pressure increase (i.e. increasing suction), and then release. The cycling vacuum reproduces baby behavior and has at least a stimulation pattern and an expression pattern; examples of such patterns are reproduced in WO 2021 160 875, WO 2010 096 547, WO 2020 178 737, WO 2008 034 193, WO 2022 132 565, WO 02 053 208 and WO 03 082 378. Each pattern is made of vacuum curves that are cyclically reproduced by the vacuum pump.

Among those WO 2010 096 547 discloses a pumping curve with fast vacuum application.

WO 2020 178 737 also teaches a pumping curve with vacuum pulsation.

WO 2008 034 193 teaches pumping curves with fast vacuum application.

WO 02 053 208 teaches a pumping curve with improved vacuum application.

Finally, WO 2022 132 565 teaches a pumping curve with pulsating fast vacuum application.

### UNDERLYING PROBLEM

The pumping curves known from the prior art do not allow optimization of the vacuum application to the breast and thus milk extraction from the breast. For this, the above prior art teaches to maintain as much vacuum as possible and generate the maximum suction/negative pressure as fast as possible.

### SUMMARY OF THE INVENTION

The present invention proposes an optimized vacuum application to the breast of a lactating user, in which the vacuum application is optimized with the scope to obtain an increase milk extraction.

It was discovered that in order to optimize vacuum application different aspects have to be taken into account.

It was in particular discovered that four phases can be identified in a pumping curve that if properly set up allow an optimal vacuum application to the breast and maximization of the time and level of vacuum that is applied to the breast.

A first phase of the suctions cycle, which is identified as a vacuum start phase of this suctions cycle gently applies vacuum to the breast, so that the breast can set in the breast shield and allows vacuum application occurring subsequently; this part is typically not useful for milk expression but is only relevant for comfort and breast preparation to the vacuum application.

A second phase of the cycle, which is identified as a vacuum application phase, applies vacuum in order to start and maintain milk expression. The slope of this second phase may be fast in order to ensure fast vacuum application and thus efficient milk extraction.

A third phase of the cycle, which is identified as a vacuum maintenance phase, maintains the vacuum substantially constant, this can be useful to increase vacuum application and thus milk extraction.

A fourth phase of the cycle, which is identified as a vacuum release phase, is less relevant for optimized milk extraction. The vacuum release phase is provided to increase the pressure and thereby reduce the vacuum to raise the pressure to the initial pressure at the beginning of the vacuum start phase. The duration of the vacuum release phase may be dominated by the inertia of the breast pump, in particular the limited capability of the breast pump with the breast shield sealed against the breast of the user to allow air to enter into the nipple tunnel of the breast shield and thus reduce the pressure for the nipple and therefore the suction pressure driving the expression of milk from the breast of the user.

As far as the present description refers to vacuum levels, it refers to the pressure within the nipple tunnel as measurable by a sensor. This does not mean that the breast pump of the present invention needs to have such a sensor. A sensor may e.g. be used to calibrate the type of the breast pump to store measured pressure values along with operational parameters of the pump to reproduce the pressure performance by applying respective operation parameters without utilizing a sensor for monitoring or controlling the resulting pressure condition. Thus, the pressure condition referred to in this specification may correspond with an actual pressure condition within the nipple tunnel and expected to be created for exposing the breast of the user to such pressure condition by controlling specific operational parameters and assuming that the breast pump will reproduce the pressure conditions assigned to those operational parameters of the pump.

### DESCRIPTION OF THE INVENTION

The breast pump has a breast shield connected to the aggregate possibly powered by a built-in battery or connection to the power via a cable. The connection between the breast shield and the pump can occur via a tube but also in-bra solutions are possible, in which the breast shield, the housing including the aggregate and the control and possibly the battery, as well as the milk container form a unitary body, which body is supported and held in place by the bra of the user.

The pump includes an aggregate for generating vacuum that is connected to the breast shield. The aggregate may comprise a motor like an electric motor and a piston e.g. a cylinder/plunger and is of e.g. multi stroke type. Thus, more than one run of the plunger is required to reach the maximum vacuum. In other words, for each run of the plunger the aggregate generates a vacuum step.

Advantageously, the pulsation is achieved via the multi stroke aggregate, so that at each run of the plunger the aggregate generates and transmits to the breast shield one vacuum step.

After the plunger has completed its run and moves back to its starting position to prepare for a subsequent run, vacuum can be slowly released, so that pulsation may be achieved.

The pump usually has a selector to select the maximum level of vacuum that is applied to the breast.

Typically, the user selects the pumping level based on comfort, health care professional's recommendation, and efficacy of milk production and the pump applies vacuum to the breast based on the selected vacuum level.

The controller is usually adapted to provide multiple vacuum curves, e.g. often 10 or more, e.g. 16. These vacuum curves are defined by a same base curve that is scaled according to different levels of maximum vacuum. For a diagram with time on the horizontal axis and vacuum of the vertical axis, scaling implies application of the same reduction coefficient on both axes.

The curves according to the invention have four phases, each with a specific scope, to
maximize the vacuum that is applied with that specific curve, i.e. for each curve the area below the curve is optimized/maximized, so that the curve with that specific maximum vacuum maximizes the area below the curve; the area below the curve is proportional to the extracted milk.
allow the user to select and use (for the whole of the pumping cycle) the curve with the highest vacuum level possible, according to her sensibility, among the vacuum levels that are available from the breast pump.

In the breast pump of the present invention the controller is adopted to control the aggregate to provide at least one cyclic suction profile consisting of a vacuum start phase, a vacuum application phase, a vacuum maintenance phase and a vacuum release phase, wherein the vacuum start phase is followed by the vacuum application phase, wherein the vacuum application phase is followed by the vacuum maintenance phase, wherein the vacuum maintenance phase is followed by the vacuum release phase, wherein the vacuum release phase defines the end of the cycle and is followed by the vacuum start phase of the next cycle.

The four phases mentioned above immediately follow each other with no other phases than the four phases mentioned above contained within the cyclic suction profile all cycle. Accordingly, the cycle starts with the vacuum start phase and terminates with the vacuum release phase, which vacuum release phase is followed by the vacuum start phase of the next cycle.

In the following, the characteristics of the different phases of the breast pump of the present invention will be described. Negative pressure gradients indicate a phase of increasing suction force within the nipple tunnel. The gradients are identified by mmHg/per second. The duration of each phase is identified in percentages relative to the total duration of the cycle. As each cycle consists of four phases, the total duration of the four phases will sum up to 100%.

### 1. Vacuum Start Phase

Here the vacuum is applied in a gentle way; this prepares the breast to the subsequent fast application and long maintenance of the vacuum, so that the user is also able to withstand high vacuum levels, i.e. she can withstand and select a pumping curve with higher vacuum level.

The vacuum start phase is defined by:
Duration of this phase is between 12-18% of the cycle and preferably 15% with some tolerance around his value. The tolerance can be, for example, +/−2% or +/−1%.
Slope of this phase is −140 to −220 mmHg/s and preferably −180 mmHg/s with some tolerance around his value. The tolerance can be, for example, +/−5%, +/−2% or +/−1%.

### 2. The vacuum Application Phase

This phase applies the vacuum in a fast way; the user can withstand it because the breast is properly set in the breast shield and is already prepared for the vacuum application.

The vacuum application phase is defined by:
Duration of this phase is between 31-48% of the cycle and preferably 39.5% with some tolerance around his value. The tolerance can be, for example, +/−5%, +/−3% or +/−2%.
Average slope of this phase is between −200 to −700 mmHg/s and preferably −470 mmHg/s with some tolerance around his value. The tolerance can be, for example, +/−40 mmHg/s and preferably +/−20 mmHg/s and more preferably +/−10 mmHg/s.

This vacuum application phase can have a first application subphase and preferably a second application subphase following the first application subphase and more preferably a third application subphase following the third application subphase; the application subphases being defined as follows.

### First Application Subphase

Duration of this subphase is between 6-12% of the cycle and preferably 9% with some tolerance around his value. The tolerance can be, for example, +/−2% or +/−1%.
Slope of this subphase is −360 to −440 mmHg/s and preferably −400 mmHg/s with some tolerance around his value. The tolerance can be, for example, +/−5%, +/−3% or +/−2%.

### Second Application Subphase

Duration of this phase is between 18-24% of the cycle and preferably 21% with some tolerance around his value. The tolerance can be, for example, +/−2% or +/−1%.
Slope of this phase is −540 to −620 mmHg/s and preferably −580 mmHg/s with some tolerance around his value. The tolerance can be, for example, +/−5%, +/−3% or +/−2%.

### Third Application Subphase

Duration of this phase is between 7-12% of the cycle and preferably 9.5% with some tolerance around his value. The tolerance can be, for example, +/−2% or +/−1%.
Slope of this phase is −240 to −320 mmHg/s and preferably −280 mmHg/s with some tolerance around his value. The tolerance can be, for example, +/−5%, +/−3% or +/−2%.

### 3. Vacuum Maintenance Phase

Vacuum maintenance phase allows maximization of the vacuum applied by the specific curve.

The vacuum maintenance phase is defined by:
Duration of this phase is between 19-25% of the cycle and preferably 22% with some tolerance around his value. The tolerance can be, for example, +/−2% or +/−1%.
Slope of this phase is between −10 mmHg/s and 10 mmHg/s, preferably with a substantially constant average pressure. Thus, in the vacuum maintenance phase the vacuum is substantially constant but may slightly increase or it can also decrease. The control is usually controlled to keep the pressure in vacuum maintenance phase constant. However, some leakage may lead to a slight increase of the suction pressure i.e. higher pressure values. Adjustment to compensate for such leakage may lead to a further and slight decrease in the pressure. The other phases may exhibit at least predominately a generally continuous slope.

### 4. Vacuum Release Phase

In this phase vacuum is released.

The vacuum release phase is defined by:
Duration of this phase is between 21-27% of the cycle and preferably 24% with some tolerance around his value. The tolerance can be, for example, +/−2% or +/−1%.
Slope of this phase is not important and may be fast, e.g. between 600 and 1000 mmHg/s. As mentioned before, the controller could be adopted to allow instantaneous relaxation of the negative pressure from the pressure value during the vacuum maintenance phase to a baseline pressure around 0 mmHg or slightly below this value, while the actual pressure condition in the nipple tunnel may more slowly adopt due to inertia of the system, in particular the reduced capability to allow ambient air to quickly enter into the nipple tunnel.

### DESCRIPTION OF THE DRAWINGS

These and other advantages of the invention will be further understood upon consideration of the following detailed description, in which:

FIG. 1 is a perspective view of an embodiment of a breast pump,

FIG. 2 is a schematic graph of different phases of a cyclic suction profile controlled by the breast pump of the present invention, and

FIG. 3 is a perspective exploded view of an alternative embodiment of a breast pump.

### DETAILED DESCRIPTION OF THE DRAWINGS

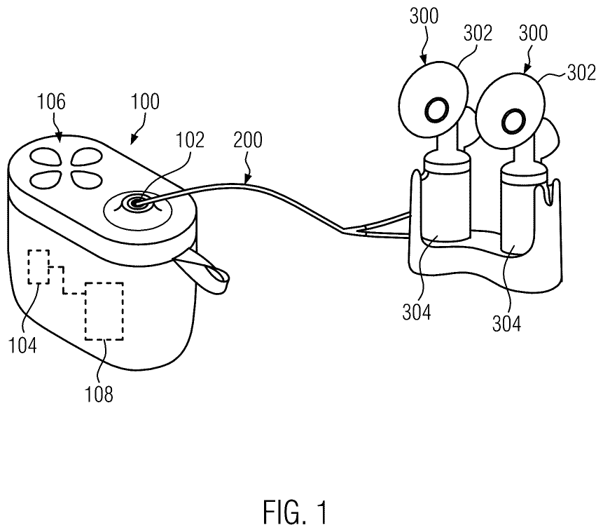

FIG. 1 shows a breast pump 100 with a drive interface 102 and a controller 104 operatively connected to a controller interface 106. The controller 104 controls an aggregate 108 for providing a cyclic suction profile at the drive interface 102. Via a tube 200 the drive interface 102 is connected to breast shield components 300 each having a breast shield 302 adapted to receive at least in part the breast of a lactating user and a reservoir 304 for receiving the expressed milk. The cyclic suction profile of the breast pump 100 will provide cyclic suction within the breast shield 302 for expressing milk from the breast of the user when using the breast pump 100.

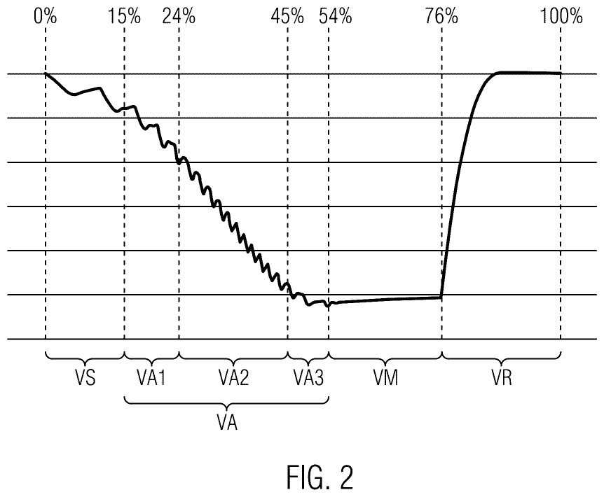

FIG. 2 shows a graph with a vacuum curve, which may be one of a set of curves available to be chosen in such a breast pump or any kind of breast pump. Each vacuum curve has a vacuum start phase VS, a vacuum application phase VA, a vacuum maintenance phase VM and a vacuum release phase VR. Those four phases are consecutive phases and exclusively define a cyclic suction profile, which cyclic suction profile starts at time 0 seconds (time at the abscissa in FIG. 2) at the beginning of the vacuum start phase VS and terminates at approximately 1.4 seconds at the end of the vacuum release phase VR.

In the first phase, i.e. the vacuum start phase VS, the pressure is reduced with an average gradient or slope of about −180 mmHg/s.

The second phase, i.e. the vacuum application phase VA, is divided into three subphases: a first application subphase VA1, a second application subphase VA2 following the first application subphase VA1, and a third application subphase VA3 following the second application subphase VA2. In the first application subphase VA1 the pressure is reduced with an average gradient or slope of about −400 mmHg/s. In the second application subphase VA2 the pressure is reduced with a more pronounced average gradient or slope of about −580 mmHg/s. In the third application subphase VA3 the pressure is reduced with an average gradient or slope of about −280 mmHg/s.

In the third phase, i.e. the vacuum maintenance phase VM, the pressure is almost constant. In fact, FIG. 2 shows a slight increase of the pressure during the vacuum maintenance phase VM.

This vacuum maintenance phase VM is followed by the vacuum release phase VR, in which the negative pressure applied during the vacuum maintenance phase VM is released to assume the initial pressure of the vacuum start phase VS, which vacuum start phase VS is the first phase of the next cycle, which cycle shows the same phases as discussed before with the same characteristics of each phase.

In the embodiment of FIG. 2, the vacuum application phase VA is the longest phase of the cycle whereas the vacuum start phase VS is the shortest phase of the cycle. The vacuum release phase VR has a longer duration than the vacuum start phase VS. The vacuum maintenance phase VM has a shorter duration than the vacuum release phase VR and a shorter duration in comparison to the vacuum application phase VA.

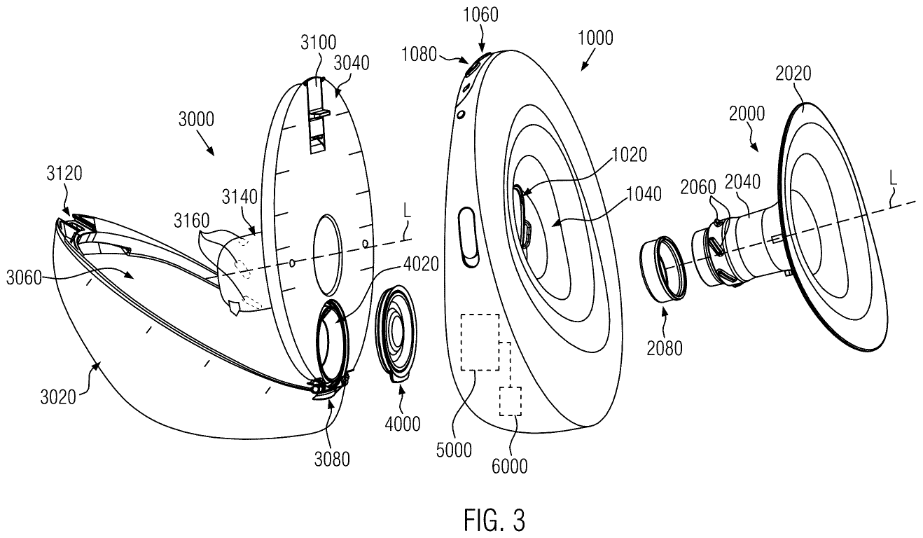

The vacuum curve shown in FIG. 2 may be implemented with any kind of breast pump. According to FIG. 3, such a breast pump—as an alternative example to the breast pump illustrated in FIG. 1—comprises a housing 1000, a breast shield element 2000 and a milk container 3000. The housing 1000 has a central opening 1020 providing a receptacle 1040. The breast shield element 2000 has a flange section 2020, which is unitarily formed with a nipple tunnel 2040 extending along a longitudinal axis L. The breast shield element 2000 is formed of a transparent material. The forward end of the nipple tunnel 2040 is provided with threads 2060 for coupling the breast shield element 2000 with the milk container 3000, after the nipple tunnel 2040 has been advanced through the receptacle 1040, such that the housing 1000 is sandwiched between the breast shield element 2000 and the milk container 3000.

The forward free end of the nipple tunnel 2040 is provided with a two-way valve 2080.

The milk container 3000 comprises a container shell element 3020 and a lid element 3040, which can be connected with each other to thereby enclose a milk chamber 3060. At a lower end the lid element 3040 is hinged to the container element 3020 via a hinge 3080. The opposite upper end is provided with a spout closure element 3100 which not only secures the lid element 3040 against the container shell element 3020 to properly enclose the milk chamber 3060, but which also provides a spout 3120 to pour out milk from the milk chamber 3060 while the container shell element 3020 is still connected with the lid element 3040.

In FIG. 3 reference numeral 1060 identifies an upper end of the housing 1000, which upper end is provided with a user interface 1080, which is directed toward the face of the user if the breast pump is assembled and laid with the flange section 2020 of the breast shield element 2000 against the breast of the user in a use position.

Between the housing 1000 and the lid element 3040 FIG. 3 shows a membrane element 4000, which is releasably connectable to the lid element 3040 to enclose a membrane working chamber 4020 within the milk container 3000. The lid element 3040 comprises a nipple tunnel receptacle 3140 projecting into the milk chamber 3060 and from a flat section of the lid element 3040. This nipple tunnel receptacle 3140 is provided with further threats 3160 adapted to cooperate with the threats 2060 of the nipple tunnel 2040 to secure the breast shield element 2000 against the milk container 3000.

The housing 1000 houses an aggregate 5000 and a control 6000, wherein the aggregate 5000 cooperates with the membrane 4000 for generating vacuum that is connected to the breast shield and wherein the control 6000 controls the aggregate 5000 for providing a cyclic suction profile. The aggregate 5000 may comprise a motor like an electric motor and a piston e.g. a cylinder/plunger and can be of e.g. multi stroke type. A battery may be provided in the housing 1000 as power source.

The above description and details elucidate the inventors' findings according to which an optimized vacuum application to the breast of a lactating user will increase milk extraction. It was in particular discovered that the four phases identified above in a properly set pumping curve/pumping cycle will allow an optimal vacuum application to the breast and maximization of the time and level of vacuum that is applied to the breast.

### LIST OF REFERENCES

100 breast pump
102 drive interface
104 controller
106 controller interface
108 aggregate
200 tube
300 breast shield components
302 breast shield
304 reservoir
1000 housing
1020 opening
1040 receptacle
1060 upper end
1080 user interface
2000 breast shield element
2020 flange section
2040 nipple tunnel
2060 threads
2080 two-way valve element
3000 milk container
3020 container shell element
3040 lid element
3060 milk chamber
3080 hinge
3100 spout closure element
3120 spout
3140 nipple tunnel receptacle
3160 threads
4000 membrane
4020 membrane working chamber
5000 aggregate
6000 control
L longitudinal axis
VM vacuum maintenance phase
VR vacuum release phase
VA vacuum application phase
VS vacuum start phase
VA1 first application subphase
VA2 second application subphase
VA3 third application subphase

What is claimed is:

1. A suction profile for a breast pump comprising a vacuum start phase, a vacuum application phase, a vacuum maintenance phase and a vacuum release phase, wherein the vacuum start phase is followed by the vacuum application phase, wherein the vacuum application phase is followed by the vacuum maintenance phase, wherein the vacuum maintenance phase is followed by the vacuum release phase, wherein the vacuum release phase defines the end of the cycle,
wherein the vacuum start phase extends over between 12 to 18% of the cycle duration and has an average pressure slope of between −140 and −220 mmHg/s,
wherein the vacuum application phase comprises a first application subphase that extends over between 6% to 12% of the cycle duration and has an average pressure slope of between −360 and −440 mmHg/s.

2. The suction profile according to claim 1, wherein the vacuum application phase comprises a second application subphase following the first application subphase, wherein the second application subphase extends over between 18% to 24% of the cycle duration and has an average pressure slope of between −540 and −620 mmHg/s.

3. The suction profile according to claim 2, wherein the vacuum application phase comprises a third application subphase following the second application subphase, wherein the third application subphase extends over between 7% to 12% of the cycle duration and has an average pressure slope of between −240 and −320 mmHg/s.

4. The suction profile according to claim 3, wherein the first application subphase extends over 9%+/−2% of the cycle duration and has an average pressure slope of −400 mmHg/s +/−5%, wherein the second application subphase extends over 21%+/−2% of the cycle duration and has an average pressure slope of −580 mmHg/s+/−5%, and wherein the third application subphase extends over 9.5%+/−2% of the cycle duration and has an average pressure slope of −280 mmHg/s +/−5%.

5. The suction profile according to claim 1, wherein the vacuum start phase extends over 15% +/−2% of the cycle duration and has an average pressure slope of −180 mmHg/s+/−5%.

6. The suction profile according to claim 1, wherein the vacuum application phase extends over 39.5%+/−3% of the cycle duration and has an average pressure slope of −470 mmHg/s+/−40 mmHg/s and preferably +/−20 mmHg/s and more preferably +/−10 mmHg/s.

7. The suction profile according to claim 1, wherein the vacuum maintenance phase extends over between 19 to 25% of the cycle duration and has an average pressure slope of between −10 mmHg/s and 10 mmHg/s and preferably the vacuum maintenance phase extends over 22%+/−2% of the cycle duration and has a substantially constant average pressure slope.

8. The suction profile according to claim 1, wherein the vacuum release phase extends over between 21 to 27% of the cycle duration and increases the pressure from the pressure at the end of the vacuum maintenance phase to the pressure at the start of the vacuum start phase, preferably the vacuum release phase extends over 24%+/−2% of the cycle duration and preferably comprises a maximum slope of between 600 and 1000 mmHg/s.

9. The suction profile according to claim 1, wherein in the vacuum application phase the pressure is reduced in steps.

10. The suction profile according to claim 9, wherein each step of the vacuum application phase is between −18 and −25 mmHg and preferably each step of the vacuum application phase is between −20 mmHg+/−1 mmHg.

11. A breast pump (100) including a breast shield (302) adapted to at least in part receive a breast of a lactating user, an aggregate (108) for generating a cyclic suction profile for expressing milk from the breast, a reservoir (304) for the expressed milk, a controller (106) for controlling the aggregate (108), wherein the controller (106) is adapted to control the aggregate (108) to provide at least one cyclic suction profile.

12. The suction profile of claim 4, wherein the average pressure slope of the first application subphase is −400 mmHg/s+/−2%, the average pressure slope of the second application subphase is −580 mmHg/s+/−2%, and the average pressure slope of the third application subphase is −280 mmHg/s+/−2%.

13. The suction profile of claim 5, wherein the average pressure slope of the vacuum start phase is −180 mmHg/s+/−2%.
